(19) United States
(12) Patent Application Publication
(10) Pub. No.: US 2015/0266018 A1
(43) Pub. Date: Sep. 24, 2015

(54) DIRECT SPECIMEN COLLECTION CASSETTE FOR AUTOMATED PROCESSING

(71) Applicant: The Bode Technology Group, Inc., Lorton, VA (US)

(72) Inventor: Jangbir S. Sangha, Overland Park, KS (US)

(21) Appl. No.: 14/662,713

(22) Filed: Mar. 19, 2015

Related U.S. Application Data
(60) Provisional application No. 61/955,436, filed on Mar. 19, 2014.

(51) Int. Cl. B01L 3/00; C12Q 1/68

(52) U.S. Cl. CPC B01L 3/508; C12Q 1/6806; B01L 3/545; B01L 2200/18; B01L 2300/041; B01L 2300/069

(57) ABSTRACT

The present disclosure relates to a device and method of securing a biological sample collected to permit use of the collected sample with automated specimen analysis devices while maintaining a chain of custody. Further, the present invention relates to an specimen collection device for collecting and tracking a collected biological sample or specimen in a manner suitable for storage and subsequent automated analysis.

### CROSS-REFERENCE TO RELATED APPLICATION

This application claims the benefit of U.S. Provisional Application Ser. No. 61/955,436, filed Mar. 19, 2014, which is hereby incorporated by reference in its entirety.

### FIELD OF THE INVENTION

The present invention relates to devices for collecting biological samples or evidence. In particular, the present invention is related to devices that allow for collecting biological samples, while later permitting extraction of portions of the sample collected for testing and also maintaining the chain of evidence, sterility, and identification of the collected sample. Several aspects of the present invention provide a device having an absorbent for collection of fluids, blood specimens, cells, or other biological material. Further, the present invention is related to devices that allow the absorbent to be processed by automated analysis equipment.

### BACKGROUND OF THE INVENTION

It has become increasingly necessary in law enforcement to collect biological samples as evidence of a crime or for use as identifying information of a particular human (e.g., DNA collection from suspects to a crime). In the collection of biological specimens, it is necessary to associate information about the subject with the specimen at the time of collection, as there is no manner for identifying a biological specimen by simple inspection. Therefore, a useful form of an evidence collection device for use with biological specimens will contain, at least, a suspect information portion for recording subject information data thereon.

The use of biological specimens as evidence further requires that the biological specimen be securely associated with the identifying information to ensure that after a specimen has been collected, the collection paper or absorbent cannot be intentionally or inadvertently switched to a different collection paper. At a minimum, a proper evidence collection device or holder should easily and clearly show that tampering was attempted either by damage to delicate indicia on the device or by overt damage to a strongly secured device. It is further beneficial if the evidence collection device can accommodate additional forms of evidence such as the application and storage of fingerprints on the evidence holder.

A particular issue in the collection of the biological sample is that the sample collection is often taken from a living, uncooperative person. Often the sample is to be taken from the mouth of the suspect in cases where a saliva or DNA specimen is needed. In this instance, the collection absorbent used will be attached to a handle or a stick. Once the specimen is on the absorbent, the handle is no longer needed. This presents a complicating factor in specimen storage and analysis due to the extra bulk and size of the handle.

The foregoing issues relate, generally, to a process referred to as evidence “chain of custody.” The term “chain of custody” encompasses the procedures and documentation used to maintain and demonstrate the chronological history or paper trail of the evidence, showing the seizure, custody, control, transfer, analysis, and disposition of physical or electronic evidence. Documentation should include, for example, name or initials of the individual collecting the evidence, each person or entity subsequently having custody of the evidence collected, dates the items were collected or transferred, agency and case number, victim's or suspect's name, and a brief description of the item. In biological samples, the principles of evidence or sample identification involved in maintaining proper “chain of custody” are especially critical as a fluid or cellular biological sample, once collected onto an absorbent presents no distinguishing characteristics. This lack of visual characteristics foils any attempt to properly identify a biological fluid or cellular sample once it has become separated from its identifying information.

Another problem or issue presented in the analysis of biological specimens is the need to extract or transfer the collected sample from the original collection absorbent and onto an alternate medium to permit analysis or testing of the specimen using the analysis equipment that is available in the selected laboratory. Often it is necessary to separate the collection absorbent from its originally obtained identifying information that is directly associated with the original collection absorbent. This separation of specimen from identifying information can lead to mistakes in associating the specimen with the correct subject or suspect or can lead to the “chain of custody” being open to question in court and a failure to prove a crime. Sample misidentification is a major source of error both in laboratory analysis and in substantiating criminal evidence.

Accordingly, a need still exists for a specimen collection device that is securely associated with the specimen collection absorbent and subject identifying information, while providing ease of use for the collecting user in the field, ease of storage, and compatibility with automated analysis equipment. Further, it would be beneficial if such device was tamper proof or tamper evident.

### SUMMARY OF THE INVENTION

The present disclosure relates to a specimen collection device useful for collecting a sample or specimen, while securely associating the specimen collection device with identification information in a user- and storage-friendly format that is compatible with automated specimen analysis equipment. Further, the devices of the present disclosure allow the direct analysis of the collected specimen and do not require a step of transferring the collected specimen prior to analysis.

The purpose of the various inventive specimen collection devices disclosed herein is to meet collection requirements which allow specimen collection to qualify for secure evidence handling and for chain of custody proof requirements in law enforcement. In particular, in an optimal situation, it is desirable that the specimen collector carrier used for adapting the sample or specimen collection device to an automated sampling system incorporate the original specimen collection absorbent and avoids the need for any transfer of the specimen onto a second absorbent or require the transfer of a bar code or other identifying material from the collection absorbent or cassette onto specimen collection device. It also is important that the absorbent or sampling paper surface, while held within the specimen collector carrier, be exposed so that small portions or circles can be punched from the paper for delivery into testing vials for downstream analysis. It is desirable that the specimen collector carrier used for adapting the specimen collection device to an automated sampling system incorporate the original collection absorbent and avoids the need for any transfer of the specimen onto a second absorbent or require the transfer of a bar code or other identifying material from the collection absorbent or cassette or sampling device onto the specimen collector device.

As described herein, this invention provides a safe, convenient and minimally labor intensive device and method for collecting and analyzing biological samples. Also, the present invention provides methods for using the collection cassette in the collection of biological samples or evidence in forms suitable for storage and subsequent analysis. Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a perspective view of a first embodiment of a cassette;

FIG. 2 is a perspective view of a second embodiment of a cassette;

FIG. 3 is a perspective view of a third embodiment of a cassette;

FIG. 4 is a perspective view of a fourth embodiment of a cassette;

FIG. 5 is a front view of a fifth embodiment of a cassette;

FIG. 6 is a perspective view of a sixth embodiment of a cassette;

FIG. 7 is a perspective view of a first embodiment of a protect apparatus;

FIG. 8 is a perspective view of a second embodiment of a protect apparatus;

FIG. 9 is a rear perspective view of a second embodiment of a protect apparatus;

FIG. 10 is a perspective view of a third embodiment of a protect apparatus;

FIG. 11 is a perspective view of a fourth embodiment of a protect apparatus;

FIG. 12 is a perspective view of a fifth embodiment of a protect apparatus;

FIG. 13 is a perspective view of a sixth embodiment of a protect apparatus;

FIG. 14 is a perspective view of the specimen collection device attached to a protect apparatus;

FIG. 15 is a rear perspective view of a specimen collector carrier;

FIG. 16 is an exploded view of the specimen collection device of FIG. 14;

FIG. 17 is an exploded view of a specimen collector carrier;

FIG. 18 is a perspective view of a specimen collector carrier;

FIG. 19 is a perspective view of a collection absorbent;

FIG. 20 is a rear perspective view of a support cap;

FIG. 21 is a perspective view of a cover for a specimen collector carrier;

FIG. 22 is a perspective view of a specimen collector carrier;

FIG. 23 is a rear perspective view of a handle;

FIG. 24 is a front perspective view of a handle;

FIG. 25 is a perspective view of a first embodiment of a specimen collection device;

FIG. 26 is a rear perspective view of a first embodiment of a cassette;

FIG. 27 is a rear perspective view of a second embodiment of a cassette;

FIG. 28 is a rear perspective view of a third embodiment of a cassette;

FIG. 29 is a perspective view of a seventh embodiment of a cassette;

FIG. 30 is a rear perspective view of a protect apparatus with a cassette having been inserted into the protect apparatus;

FIG. 31 is a rear perspective view of a seventh embodiment of a protect apparatus;

FIG. 32 is a perspective view of an eighth embodiment of a cassette containing USB;

FIG. 33 is a perspective view of an eight embodiment of a cassette showing USB in open position;

FIG. 34 is a rear perspective view of a second embodiment of a specimen collection device;

FIG. 35 is a front perspective view of an identification card;

FIG. 36 is a rear perspective view of an identification card; and

FIG. 37 is an exploded view of an alternative embodiment of a specimen collector carrier.

### DETAILED DESCRIPTION

The present invention relates to a sample or specimen collection device 50 for the collection of biological samples in a form suitable for storage and subsequent automated analysis and a method for using. The methods of use provide a safe, convenient, easy-to-use, and reliable means for storing collected biological samples or evidence in a manner that meets chain-of-custody requirements and provides secure evidence handling, as well as reliable accuracy of analytical results. Moreover, the invention provides for enhanced convenience and efficiency when samples are analyzed using automated systems.

In several places throughout the present specification, guidance is provided through example embodiments. In each instance, the recited embodiments serve only as representative groups and are not meant to be exclusive.

Referring to the various figures, the specimen collection device 50 generally comprises a cassette 10 and a specimen collector carrier 30, wherein the specimen collector carrier 30 is removably attached to the cassette 10.

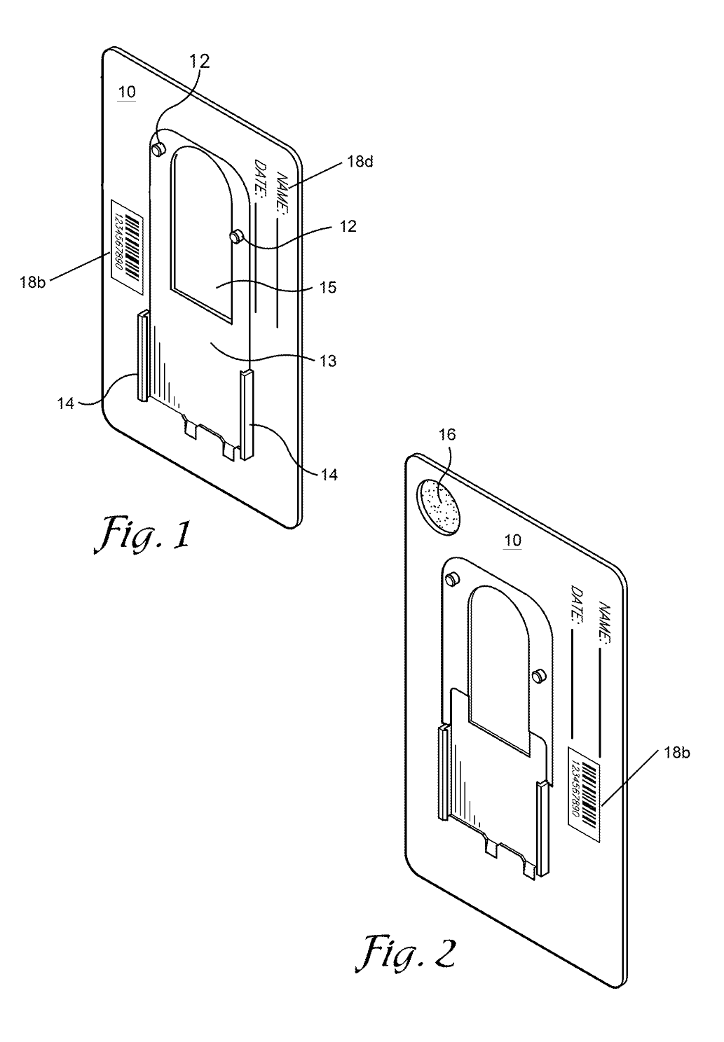

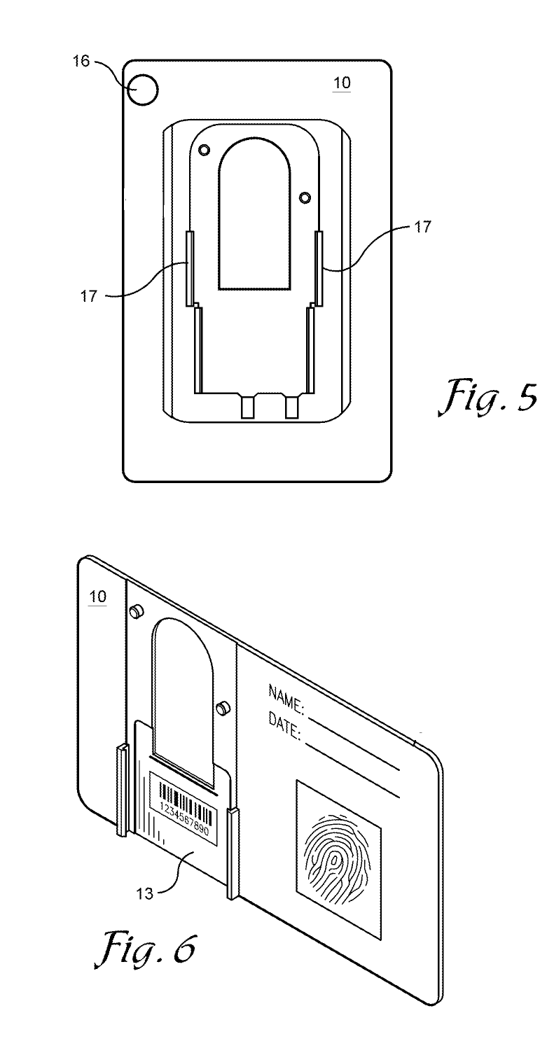

The collection cassette of the invention provides for storage and subsequent automated analysis of the stored biological sample or evidence. Referring to FIGS. 1-6, the cassette 10 generally comprises a rectangular shape. In some aspects, the cassette 10 is shaped similar to a standard credit card, having a length about 85 mm, a width about 54 mm, and a thickness of about 1 mm. In some aspects, the cassette 10 has a length of about 30, 35, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 mm or more. In some aspects, the cassette 10 has a width of about 30, 35, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 mm or more. In some aspects, the cassette 10 has a thickness of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0 mm or more. In some aspects, the cassette 10 has a length ranging from about 30 mm to about 90 mm. In some aspects, the cassette 10 has a width ranging from about 30 mm to about 90 mm. In some aspects, the cassette 10 has a thickness ranging from about 0.1 mm to about 3.0 mm. In some aspects, the cassette 10 has a length ranging from about 50 mm to about 85 mm, a width ranging from about 50 mm to about 85 mm, and a thickness ranging from about 0.5 mm to about 2.5 mm. In some aspects, the cassette 10 has a thickness of about 0.8 mm to about 1.5 mm, a length of about 80 mm to about 85 mm, and a width of about 50 mm to about 55 mm. In some aspects, the cassette 10 has a thickness of about 0.8 mm to about 1.5 mm, a length of about 50 mm to about 55 mm, and a width of about 50 mm to about 55 mm. In some aspects, the cassette 10 has a length of about 53 mm and a width of about 53 mm. In some embodiments, the cassette 10 has a length of about 85 mm and a width of about 53 mm. The cassette shape may be optimized for use in automated handling. As is apparent from the figures provided, the cassette 10 may be vertically configured, as illustrated in FIGS. 1-3 and 5, or it may be horizontally configured as shown in FIGS. 4 and 6.

In certain embodiments, the cassette 10 includes a receiver impression 13, as shown for example in FIGS. 1 and 6. The receiver impression 13 includes a collector attachment means. The collector attachment means may be any attachment means known in the art that secures the specimen collector carrier 30 to the cassette 10 such that the collection absorbent 22 aligns with the receiver impression 13. Such collector attachment means include, without limitation, a plurality of pegs and respective voids for press fitting the specimen collector 30 to the cassette 10. In some aspects, the receiver impression 13 includes a set of attachment pegs 12. In other aspects, the receiver impression 13 includes a set of collector attachment voids. In other aspects, the receiver impression 13 includes a combination of collector attachment voids and collector attachment pegs.

In preferred embodiments, the receiver impression 13 is aligned with at least one collector retaining flange 14, as shown in FIGS. 1 and 2. The collector retaining flange assists in aligning the specimen collector carrier 30 with the receiver impression 13 of the cassette, as well as securing the attachment of the specimen collector carrier 30 to the cassette 10. In alternative embodiments, one or more of the retaining flanges may be L-shaped such that the retaining flange(s) is partially extended along the bottom of the specimen collector carrier 30 once it is inserted into the cassette 10 to prevent the specimen collector carrier from sliding out of the cassette.

In some aspects, the receiver impression 13 includes an access void 15 in unobstructed registration with the collection absorbent 22 when a specimen collector carrier 30, is attached to the cassette 10. The access void 15 of the cassette 10 may be covered or encased in a protective material, for example, a plastic film, which may further protect against degradation and contamination of collected biological samples. Suitable plastic films or protective material are known in the art and may include polystyrene, polyethylene, polypropylene and other suitable lamination plastics.

In certain embodiments, the cassette 10 includes tamper resistant attachment means to attach the specimen collector carrier 30 to the cassette such that any attempt to separate the specimen collector carrier from the cassette is prevented or indicated. Such tamper resistant attachment may include such tamper resistant attachment means known in the art, including but not limited to tamper resistant seals, tampering indication dyes, and tamper resistant snaps. In some aspects, the cassette 10 includes one or more tamper resistant snaps 17, as shown in FIG. 5. Such tamper resistant snaps 17 allow the specimen collector carrier 30 to be press-fit into the cassette 10 so that the removal of the specimen collector carrier from the cassette will be hindered or thwarted without causing noticeable damage to the specimen collection device 50, the cassette 10, and/or the specimen collector carrier 30.

In certain embodiments, the cassette 10 may include a clean punch area 16, as illustrated in FIGS. 2-5. Suitable clean punch areas provide a material that is used as the collection absorbent, but is free from collected specimen. The clean punch area is used to clean a punch apparatus between punches taken from the collection absorbent 22 for downstream specimen analysis. The clean punch area 16 may be located anywhere on the cassette 10 that is compatible for use by automated analysis equipment and that does not interfere with the collection absorbent 22 or identification indicia.

The specimen collector carrier 30 as depicted in FIGS. 15, 17, and 18 first includes a collection absorbent 22 capable of attaching to at least one biological sample or evidence and for storing such biological sample or evidence. FIG. 19 illustrates the collection absorbent 22, which may be made from any material to which biological samples or evidence will sorb and which does not inhibit storage or subsequent analysis of the biological sample or evidence. In one aspect, the collection absorbent 22 is of a porous nature to provide entrainment of macromolecules of the biological sample or evidence onto the collection absorbent 22. Material suitable for this purpose includes, but is not limited to, a matrix which is cellulose based (e.g., cellulose, nitrocellulose or carboxymethylcellulose), hydrophilic polymers including synthetic hydrophilic polymers (e.g., polyester, polyamide, carbohydrate polymers), polytetrafuroethylene, fiberglass, and porous ceramics.

Additives may be included on the collection absorbent 22 or added to the collection absorbent after the specimen has been collected. The additives may be located in the receiver impression 13 of the cassette 10, or on a film or covering that traverses the access void 15 of the cassette such that when the specimen collector carrier 30 is attached to the cassette 10, the additives are placed in contact with the collection absorbent 22. In some aspects, the additives may be administered directly to the collection absorbent 22 by automatic or mechanical means. The additives may be in liquid, solid, semi-solid, or film form.

The additives may include additional compositions to preserve the biological sample or evidence, that are useful in downstream analyses, or inactivate potential pathogens. By way of example, additives may include one or more of a weak base, a chelating agent, a protein denaturant such as an anionic detergent, a surfactant, or free radical traps such as uric acid or a urate salt. Suitable weak bases include organic and inorganic bases.

Suitable inorganic weak bases include, for example, an alkali metal carbonate, bicarbonate, phosphate or borate (e.g., sodium, lithium, or potassium carbonate). Suitable organic weak bases include, for example, tris-hydroxymethyl amino methane (Tris), ethanolamine, triethanolamine and glycine and alkaline salts of organic acids (e.g., trisodium citrate). A preferred organic weak base is a weak monovalent organic base, for example, Tris. The Tris may be either a free base or a salt, for example, a carbonate salt.

Suitable chelating agents include strong chelating agents, such as those that bind multivalent metal ions with a comparable or better affinity than ethylene diamine tetraacetic acid (EDTA). A preferred chelating agent according to the invention is EDTA. One function of the chelating agent is to bind divalent ions, which, if present with the biological sample, may cause damage to the biological sample during storage. Ions which may be chelated by the chelating agent include divalent active metal ions, for example, magnesium and calcium, and transition metal ions, for example, iron. Both calcium and magnesium are known to promote degradation of genetic material by acting as co-factors for enzymes which may destroy genetic material (e.g., nucleases). In addition, transition metal ions, such as iron, may readily undergo oxidation and reduction and damage genetic material by the production of free radicals or by direct oxidation.

Additives may include an anionic detergent or surfactant. Any anionic surfactant which binds to and denatures proteins may be suitable for the invention. A preferred anionic detergent is a strong anionic detergent. As used herein, a “strong” anionic detergent includes a hydrocarbon moiety, aliphatic or aromatic, containing one or more anionic groups. Particularly preferred anionic detergents suitable for the invention include sodium dodecyl sulphate (SDS) and sodium lauryl sarcosinate (SLS). In a preferred embodiment, the anionic detergent of the invention causes inactivation of most microorganisms which have protein or lipids in their outer membranes or capsids, for example, fungi, bacteria or viruses. This includes microorganisms which may be pathogenic to humans and are present in a biological sample.

Additives may include additional components which function in subsequent analysis to be performed on the collected biological samples or evidence. Subsequent analysis which may be performed includes methods and techniques known in the art. Such analyses include gel electrophoresis, polymerase chain reaction (PCR) based analysis, reverse transcriptase initiated PCR, ligase chain reaction (LCR), DNA or RNA hybridization techniques, restriction fragment length polymorphism (RFLP), sequencing, direct sequencing, enzymatic assays, affinity labeling, methods of detection using labels or antibodies, and other methods known in the art or yet to be discovered.

The collection absorbent 22 is attached to the specimen collector carrier 30 such that the collection absorbent 22 is locked into the specimen collector carrier 30 and cannot be removed. In some aspects, the attachment means is tamper proof which causes detectable damage if the collection absorbent has been tampered with. Suitable attachment means include those that prevent the collection absorbent 22 from being detached from the specimen collector carrier 30 once it is initially attached. For example, an attachment means may include sonic welding of the collection absorbent 22. In another aspect, the attachment means includes a set of support pegs 24 and a support cap 24 (as shown in FIG. 20), with the support cap 25 having voids that register with the support pegs 24. Further, the support cap 25 may be attached to the support pegs 24 by sonic welding.

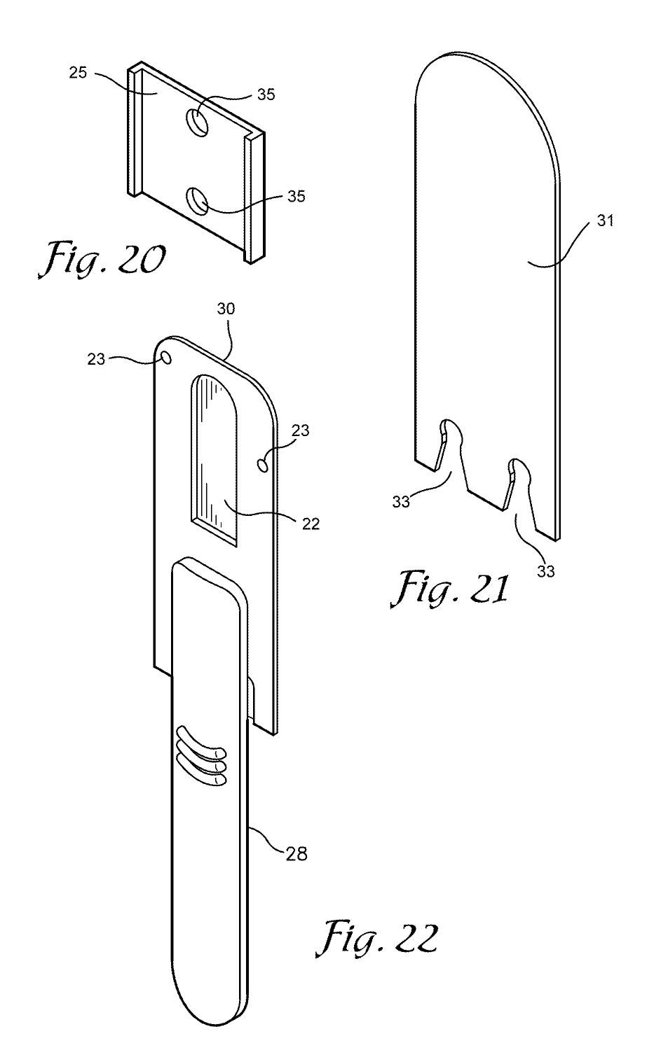

The specimen collector carrier 30 may additionally include a cover 31 for protecting the collection absorbent 22, which is illustrated in FIG. 21. The cover 31 attaches to the specimen collector carrier 30 through an attachment means. The attachment means may be any attachment means known in the art that can removably connect the cover to the specimen collector carrier 30. By way of example, without limitation, the attachment means may be by a set of cover attachment voids 33 that press-fit onto the support cap 25 attachment means. The cover 31 may be used by the user to press down on the collection absorbent 22 in order to press-fit the specimen collector carrier 30 into the cassette 10. Also, the cover 31 may be used by the user to press the collection absorbent 22 into contact with a preservative substance. Accordingly, the cover 31 allows a user to press the collection absorbent 22 without introducing possible contamination to the collection absorbent.

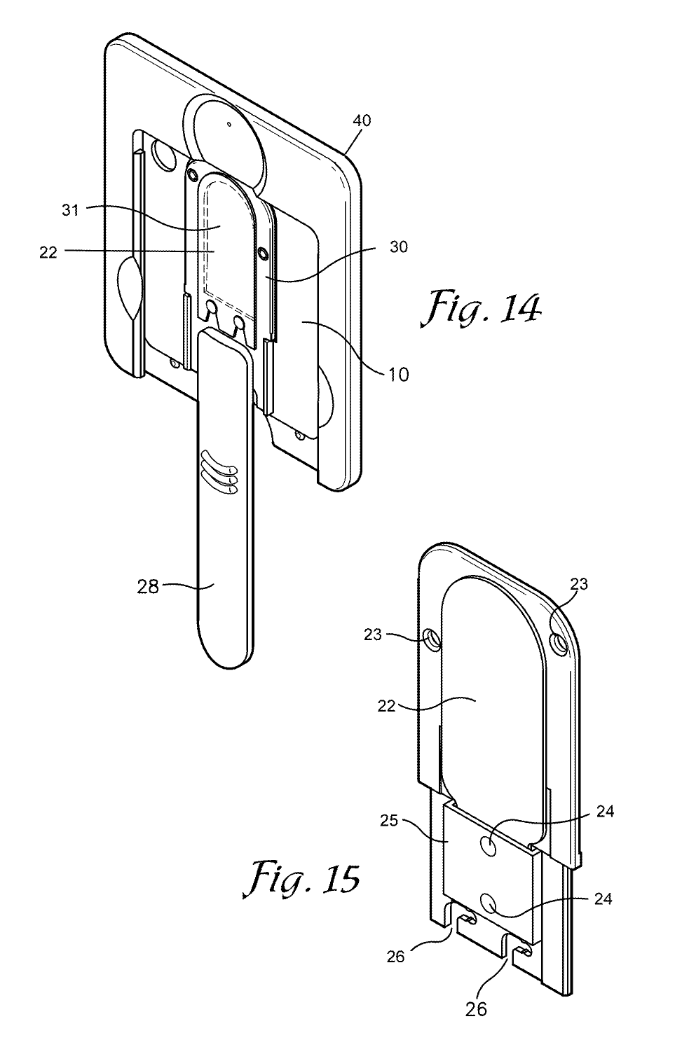

With reference to FIGS. 14, 16, 17, and 22-24, the specimen collector carrier 30 may further comprise a handle 28. The handle 28 may be removably connected to the specimen collector carrier 30 by a carrier attachment means. The carrier attachment means may be any attachment means known in the art that can secure the handle 28 to the specimen collector carrier 30. Such carrier attachment means includes, without limitation, a plurality of pegs and respective voids or slots. In some aspects, the specimen collector carrier 30 includes a plurality of handle slots 26. In some aspects, the handle 28 includes a plurality of carrier attachment pegs 29. In some aspects, the specimen collector carrier 30 includes a plurality of carrier attachment pegs, such as a first carrier attachment peg and a second carrier attachment peg. In some aspects, the handle 28 includes a plurality of handle slots 26. In other aspects, the specimen collector carrier 30 includes a combination of carrier attachment pegs and handle slots. In some aspects, the handle 28 includes a combination of carrier attachment pegs and handle slots. In some aspects, the carrier attachment pegs are tapered or beveled such that the pegs can only exit the recipient handle slot at the entry point of the handle slot. In particular, the handle slot width is smaller than the largest diameter of the peg and larger than the smallest diameter of the peg such that the peg can slide along the slot.

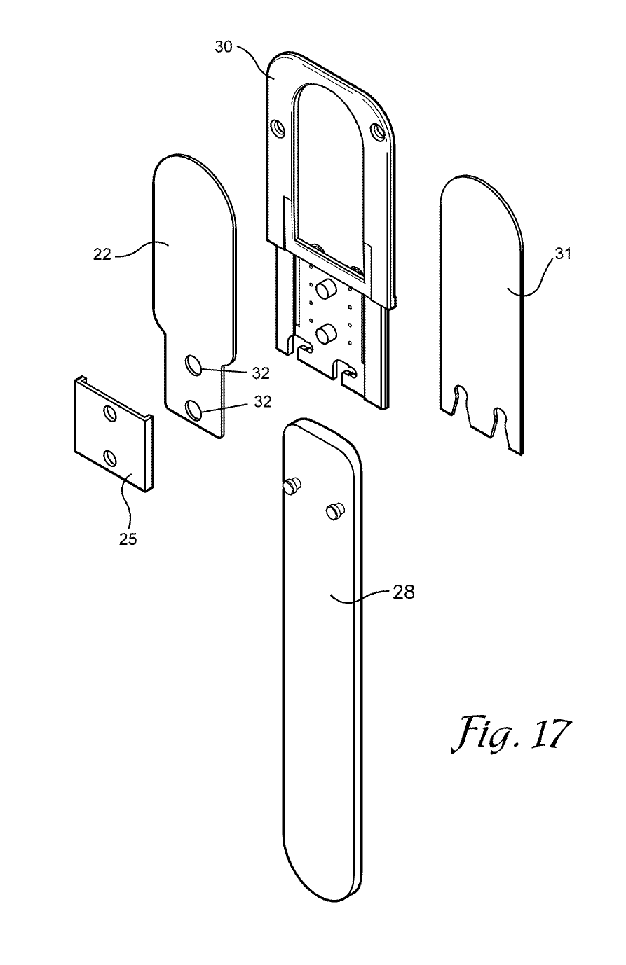

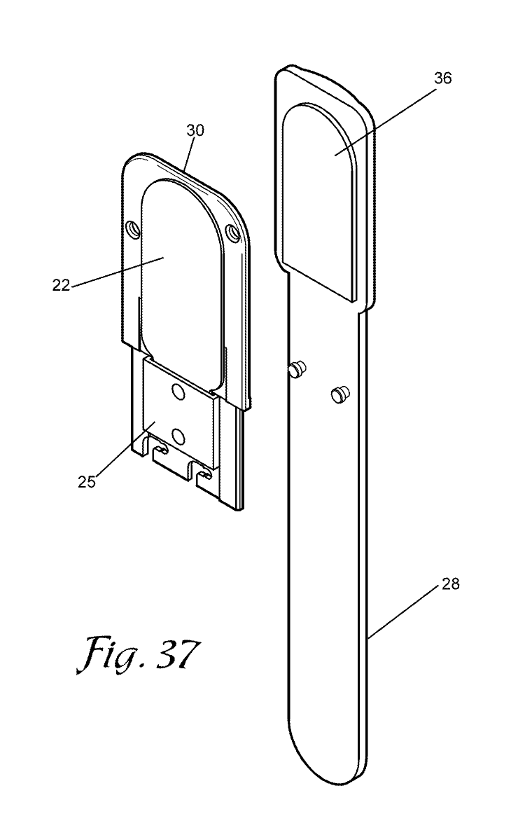

The specimen collector carrier 30 is configured such that the user can hold the handle 28 and insert the specimen collector carrier 30 into a subject's oral cavity without the user's fingers or thumbs entering the subject's oral cavity. The rigidity of the handle 28, is configured such that the specimen collector carrier 30 can be passed along the inner oral cavity multiple times allowing pressure to be applied without user appendage penetration in the oral cavity. FIG. 17 shows that the specimen collector carrier 30 may be removably connected to the top portion of the handle 28. In an alternative embodiment, illustrated in FIG. 37, the specimen collector carrier 30 comprises a handle 28 of sufficient length to fully collection absorbent 22 upon attachment to the handle 28 and includes as part of the handle 28 a collection absorbent platform 36. The collection absorbent platform 36 provides support to the collection absorbent 22 when the specimen collector carrier 30 is inserted into a subject's oral cavity to collect a sample from the subject.

FIG. 24 illustrates that the handle 28 may include holding ridges 34 to aid the user in holding the handle 28 of the specimen collection device 50. Such holding ridges may be any means known in the art for providing friction between the handle 28 and a user's appendage. By way of example, without limitation, the holding ridges 34 may be a series of ridges or bumps that are raised above the surface of the handle 28.

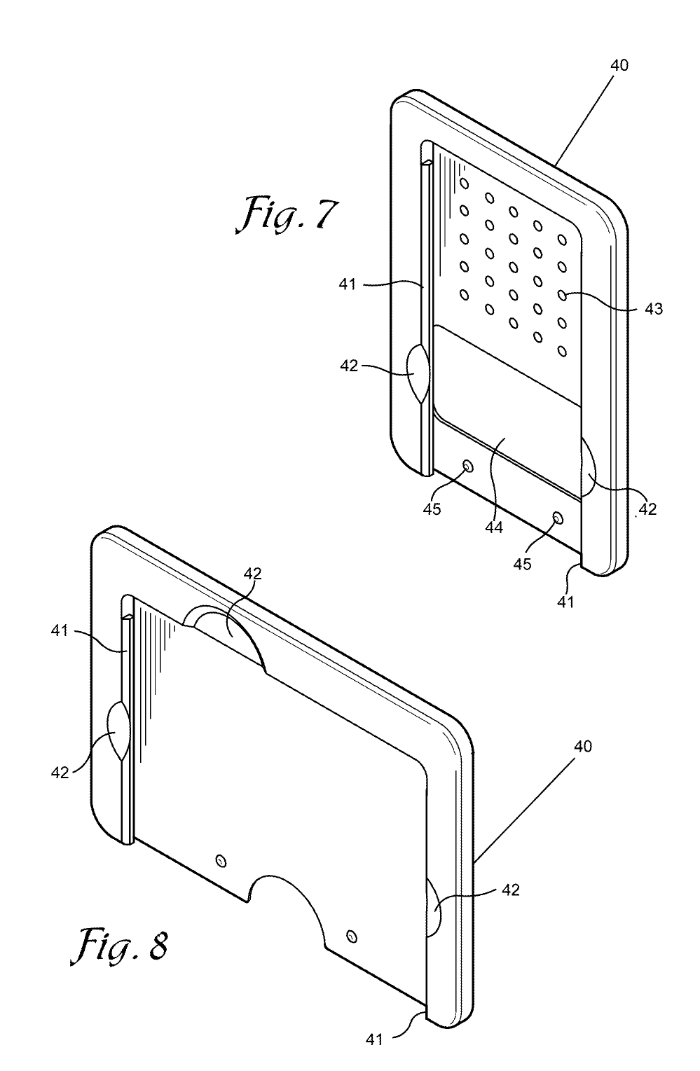

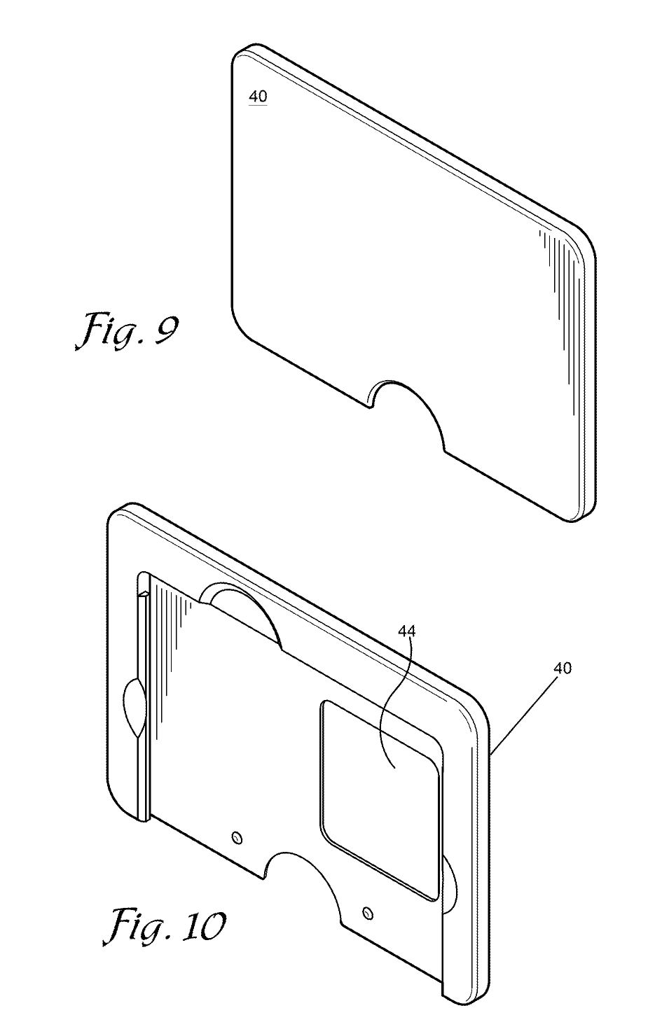

Referring now to FIGS. 7-13, the specimen collection device 50 described herein may be attached to a protect apparatus 40. Such protect apparatus 40 protects the specimen collection device 50, as illustrated in FIG. 14. The protect apparatus 40 generally will comprise a rectangular shape but may be any shape which will allow the cassette 10 to fit securely within the protect apparatus 40. In certain embodiments, the protect apparatus 40 is rectangular or square shaped, having a length about 85 mm, a width about 62 mm, and a thickness ranging from about 1 mm to about 1.13 mm. In some aspects, the protect apparatus 40 has a length of about 30, 35, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 mm or more. In some aspects, the protect apparatus 40 has a width of about 30, 35, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 mm or more. In some aspects, the protect apparatus 40 has a thickness of about 0.1, 0.2, 0.3, 0.4, 0.5, 0.6, 0.7, 0.8, 0.9, 1.0, 1.1, 1.13, 1.15, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0 mm or more. In some aspects, the protect apparatus 40 has a length ranging from about 30 mm to about 90 mm. In some aspects, the protect apparatus 40 has a width ranging from about 30 mm to about 90 mm. In some aspects, the protect apparatus 40 has a thickness ranging from about 0.1 mm to about 3.0 mm. In some aspects, the protect apparatus 40 has a length ranging from about 50 mm to about 85 mm, a width ranging from about 50 mm to about 85 mm, and a thickness ranging from about 0.5 mm to about 2.5 mm. In some aspects, the protect apparatus 40 has a thickness of about 0.8 mm to about 1.5 mm, a length of about 80 mm to about 85 mm, and a width of about 50 mm to about 55 mm. In some aspects, the protect apparatus 40 has a thickness of about 0.8 mm to about 1.5 mm, a length of about 50 mm to about 55 mm, and a width of about 50 mm to about 55 mm. In some aspects, the protect apparatus 40 has a length of about 53 mm and a width of about 53 mm. In some aspects, the protect apparatus 40 has a length of about 85 mm and a width of about 53 mm. The protect apparatus 40 shape may be optimized for use in automated handling of the cassette 10.

The protect apparatus 40 includes at least one protect flange 41. The protect flange 41 allows the cassette 10 to slide into the protect apparatus 40 and prevent the cassette 10 from separating from the protect apparatus 40. Additional means for securing the cassette 10 to the protect apparatus may be used and include at least one protect attachment means 45 to prevent the cassette 10 from slipping down and out of the protect apparatus 40 after it has been secured to the protect flanges 41. The protect attachment means 45 may be a peg, void, or any other attachment means known in the art. In some aspects, the protect apparatus 40 includes at least one protect attachment peg. In some aspects, the protect apparatus 40 includes at least one protect attachment void. In some aspects, the protect apparatus 40 includes at least one protect attachment peg and at least one protect attachment void. The protect attachment means 45 may be located on the cassette 10, the protect apparatus 40, or both. In a certain embodiment, the protect attachment means 45 is located on the inside of the protect apparatus 40, as shown in FIGS. 7,8 and 10-13.

The protect apparatus 40 also has an identification void 44. The identification void 44 allows the user to add identification information directly to the cassette 10 without contaminating the collected sample located in the collection absorbent 22. The identification void 44 also allows the user to view the identification information.

In some aspects, the protect apparatus 40 includes ventilation means 43. The ventilation means may be holes, vents, or any other ventilation means known in the art that allows air to dry the collection absorbent 22.

In some aspects, the protect apparatus 40 includes thumb indents 42. The thumb indents 42 are grasped by the user during the removal of the specimen collection device 50.

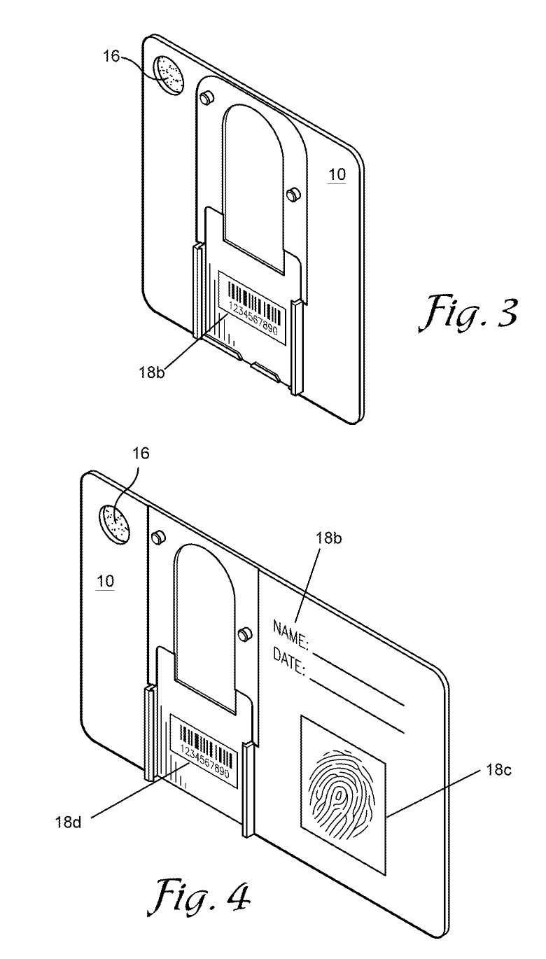

In certain embodiments, the specimen collection device 50 additionally includes identification indicia, as illustrated in FIGS. 1-4 and 6. Suitable identification indicia includes any such indicia that associates the collected biological sample, specimen or evidence with a unique identifier so that the collected biological sample, specimen or evidence may be tracked and accounted for. Examples of identification indicia may include but are not limited to bar codes, identification numbers, thumbprints, fingerprints, numbers, letters, combinations of numbers and letters, and any combination of the foregoing. For exemplary purposes, FIGS. 1 and 2 illustrate the cassette 10 having a bar code 18b as well as handwritten identification 18d. Alternatively, FIG. 3 illustrates the cassette 10 having only bar code 18b identification. In another embodiment, FIG. 4 illustrates the cassette 10 having bar code 18d, handwritten 18b, and fingerprint 18c identification.

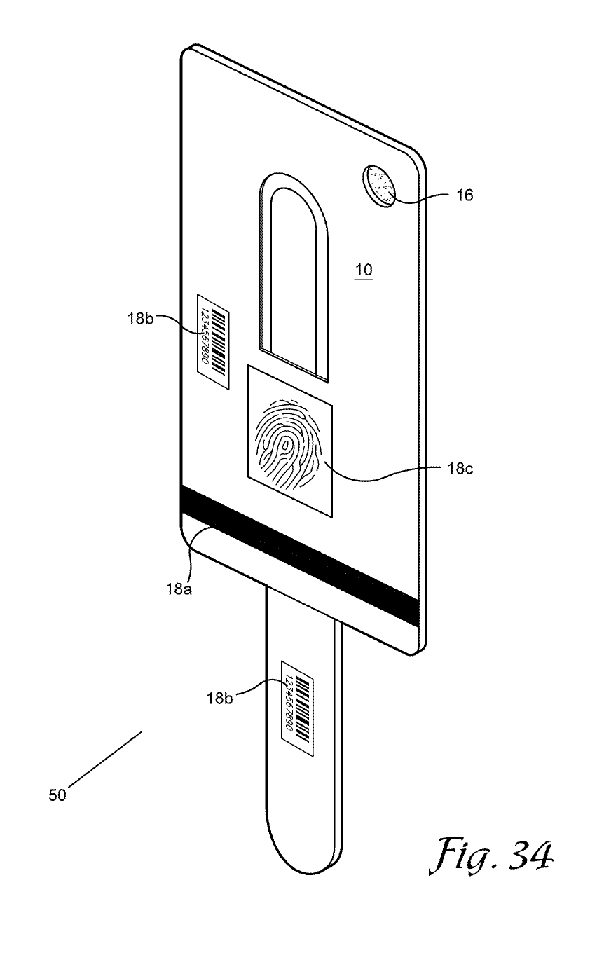

The identification indicia may be included on one or multiple components of the specimen collection device 50. For instance, the same identification indicia may be on the cassette 10, the specimen collector carrier 30 (as shown in FIG. 34), and/or the protect apparatus 40 (as shown in FIG. 31). The identification indicia may be placed anywhere on the components so long as it that does not interfere with the collection absorbent 22. Also, the identification indicia may be unique from other identification indicia fixed to the device. For instance, the components of the specimen collection device (specimen collector carrier and cassette) may each have the same bar code affixed thereon during manufacturing or packaging, and an additional identification indicia may be affixed by the analysis lab upon receipt. The specimen collection device 50 described herein provides ample surface area to which multiple identification indicia may be affixed or thereon. The identification indicia or information may be associated to an identification form.

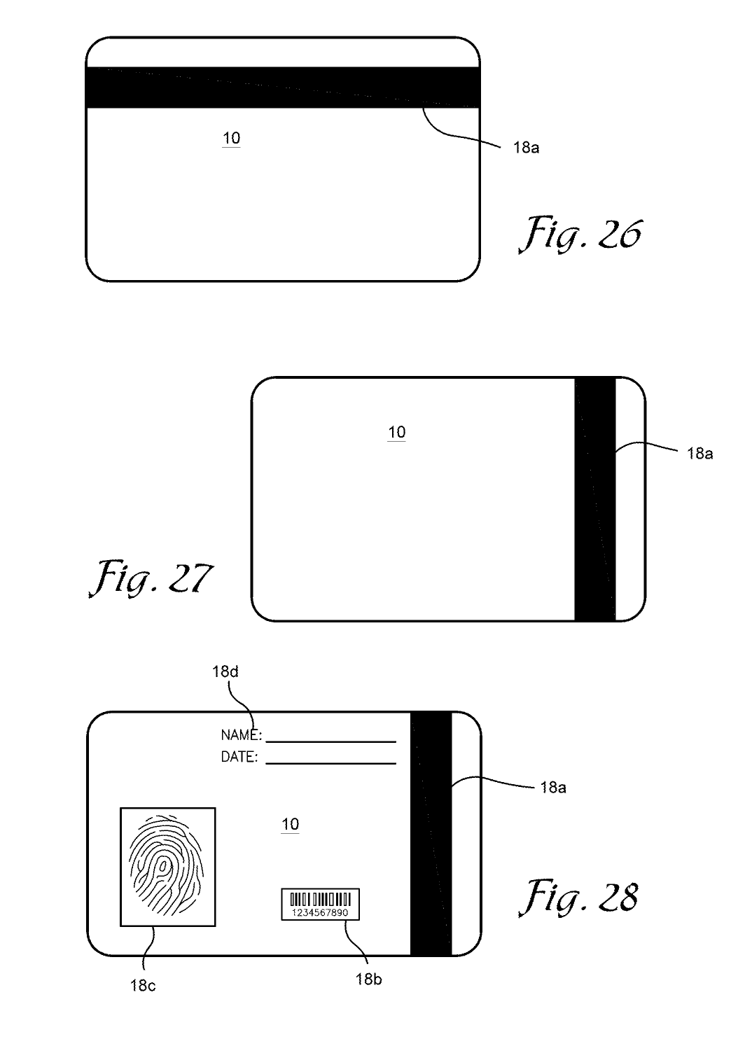

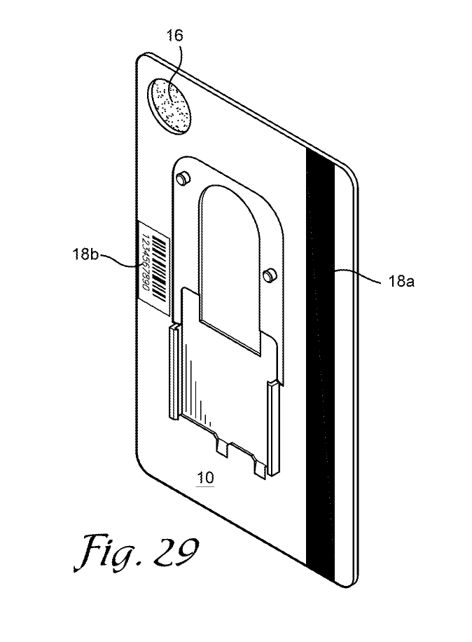

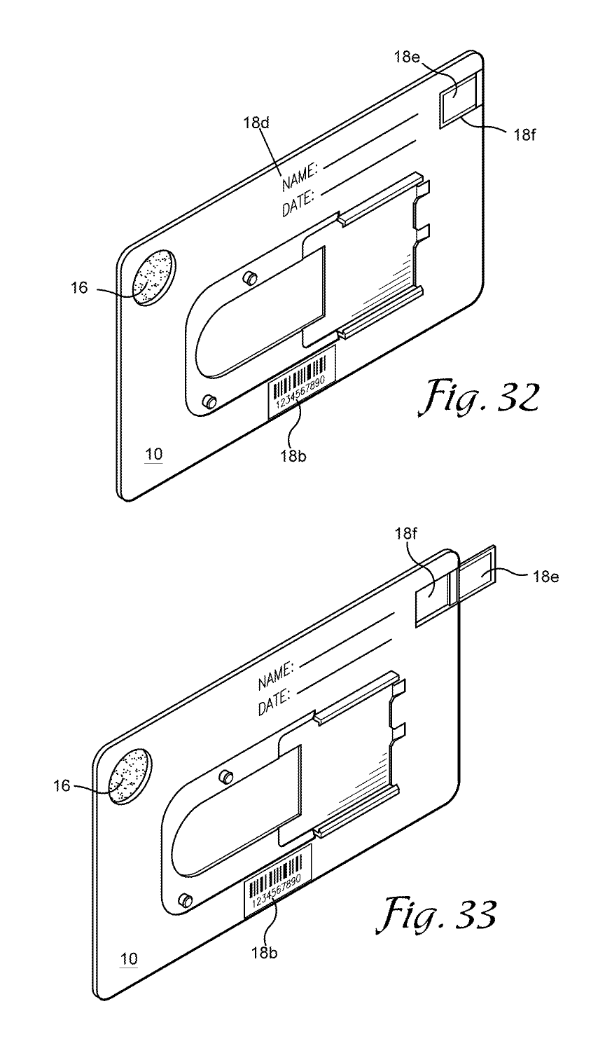

The identification indicia may be encoded in a bar code, magnetic strip, radio frequency identification tag (RFID), universal serial bus (USB) means, or other means known in the art for carrying identification information. FIGS. 26-29 illustrate a cassette 10 comprising a magnetic encoding strip 18a on the cassette's rear surface. FIG. 28 show the combination of a magnetic encoding strip 18a with bar code 18b, fingerprint 18c, and handwritten 18d identification, whereas FIG. 29 illustrates a cassette comprising a magnetic encoding strip 18a and bar code 18b identification. FIGS. 32 and 33 illustrate a cassette 10 having a USB plug 18e. The USB plug 18e may be contained in a USB retaining slot 18f until it is needed for connection to a computer or other device containing a USB socket.

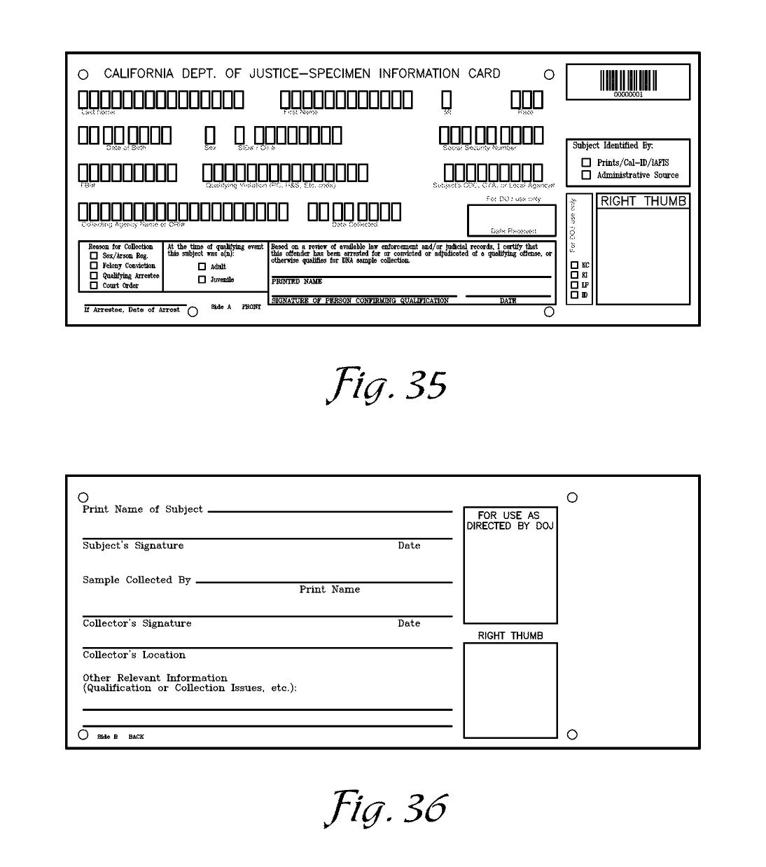

The biological sample or evidence collection device described herein may be provided with an identification card, as shown in FIGS. 35 and 36. Suitable identification cards will associate the device with the identification card by way of identical identification indicia. The identification card provides a user with a means to record important identification information that is associated with the device. The device described herein may be used with or without the identification card.

FIGS. 1-37 relate to various embodiments of the specimen collection device 50 or aspects thereof. First referring to FIG. 1, an embodiment of a cassette 10 is shown wherein the cassette has a vertical orientation and a receiver impression 13 therein, with the receiver impression 13 having at least one collector attachment peg 12. Further, FIG. 1 shows the receiver impression 13 is aligned with at least one collector retaining flange 14. The receiver impression 13 also includes an access void 15. In addition, FIG. 1 illustrates identification indicia placed on the cassette, specifically, handwritten identification 18d and a barcode 18b.

FIG. 2 is an embodiment of a cassette 10, further contains a clean punch area 16. The depicted cassette 10 has a size of 54 mm×51 mm.

FIG. 3 is an embodiment of a cassette 10 having a size of 51 mm×51 mm and a clean punch area 16;

FIG. 4 is an embodiment of a cassette 10 having horizontal orientation and a size of 85 mm×51 mm with the receiver impression 13 offset from the center position. Further, FIG. 4 illustrates the cassette with a clean punch area 16.

FIG. 5 is an embodiment of a cassette 10 having a receiver impression 13 comprising at least one tamper resistant snap 17 and a clean punch area 16.

FIG. 6 is an embodiment of a cassette 10 having a receiver impression 13 offset from the center and allowing 1 cm of clearance on each side of the receiver impression 13 for automated handling of the cassette.

FIG. 26 illustrates a cassette 10 having a magnetic encoding strip 18a positioned horizontally across the back of the cassette 10.

FIG. 27 illustrates a cassette 10 having a magnetic encoding strip 18a positioned vertically across the cassette 10.

FIG. 28 illustrates a cassette 10 having magnetic encoding strip 18a, bar code identity 18b, fingerprint 18c, and handwritten identification 18d.

FIG. 29 illustrates a cassette 10 having a clean punch area 16, as well as a magnetic strip 18a and bar code identity 18b.

FIG. 32 is an embodiment of a cassette 10 having a clean punch area 16 and identification indicia, specifically handwritten identification 18d and a universal serial bus device (USB) 18e. The USB 18e may swivel to an open position and a closed position. FIG. 32 shows a cassette 10 having a USB 18e in the closed position.

FIG. 33 is shows a cassette 10 having a USB 18e in open position, revealing a USB retaining slot 18f for storage of the USB when not in use.

FIG. 15 is a specimen collector carrier 30 having a collection absorbent 22 and a means for attaching the collection absorbent 22 to the specimen collector carrier 30. The aspect depicted in FIG. 15 shows an attachment means having at least one support peg 24, and a support cap 25, with the support cap 25 having voids that register with the at least one support peg. Further, FIG. 15 shows the specimen collector carrier 30 having at least one cassette attachment void 23, which registers with the at least one collector attachment peg 12 of the cassette 10 when the specimen collector carrier 30 is superimposed onto the cassette 10. Also, FIG. 15 shows the specimen collector carrier 30 having at least one handle slot 26.

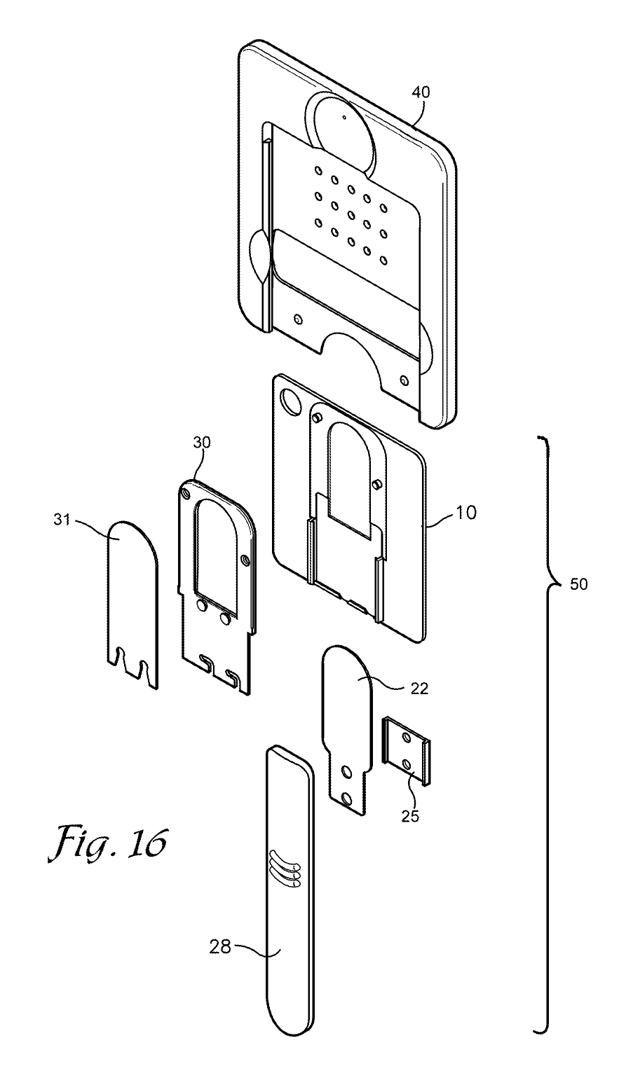

FIG. 16 illustrates the various aspects of the specimen collection device 50, including the cassette 10, the specimen collector carrier 30, the collection absorbent 22, support cap 25, cover 31, and handle 28. FIG. 16 further illustrates the protect apparatus 40.

FIG. 17 illustrates the various aspects of the specimen collector carrier 30 including the collection absorbent 22, support cap 25, cover 31, and handle 28. The collection absorbent 22 is attached to the specimen collector carrier 30 by an attachment means using the support cap 25. The handle 28 is attached to the specimen collector carrier 30 and the cover 31 is also attached to the specimen collector carrier 30 such that the collection absorbent 22 is covered by the cover 31.

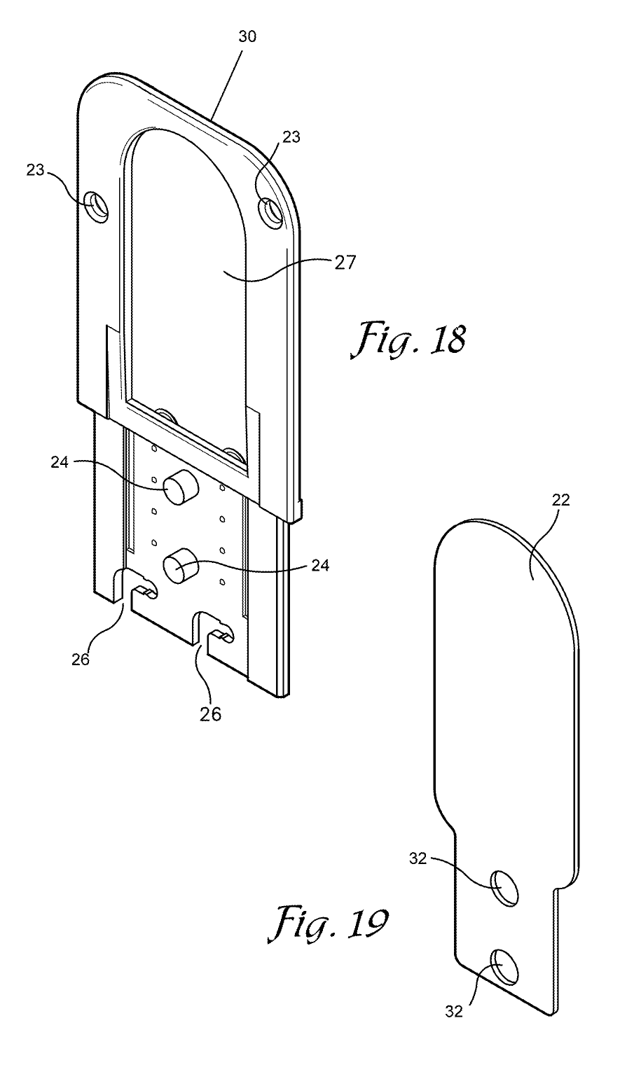

FIG. 18 illustrates a specimen collector carrier 18 having at least one cassette attachment void 23, as well as at least one handle slot 26. FIG. 18 also shows the specimen collector carrier 30 having a sample access void 27. Further, FIG. 18 shows the specimen collector carrier 30 having at least one support peg 24.

FIG. 19 illustrates a collection absorbent 22 having at least one support peg void 32.

FIG. 20 illustrates a support cap 25 having at least one support peg void 32.

FIG. 21 illustrates a cover 31 having at least one cover attachment means 33.

FIG. 22 illustrates a specimen collector carrier 30 having a handle 28 attached, where the specimen collector carrier 30 includes at least one cassette attachment void 23 and a collection absorbent 22.

FIG. 37 is an alternative embodiment of a specimen collector carrier 30, wherein the handle is of a sufficient length to provide a support for the collection absorbent 22 when the collection absorbent is aligned with the collection absorbent platform 36 at the top of the handle 28. This design allows for the specimen collector carrier 30 to be placed inside the oral cavity of a subject for sample collection while the handle 28 is fully supporting the collection absorbent 22.

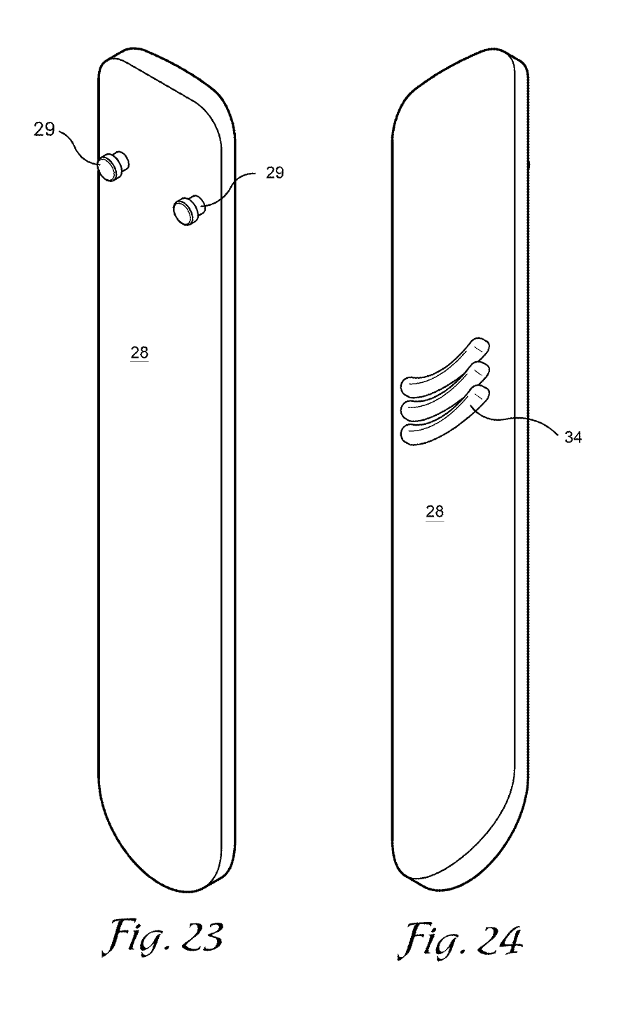

FIG. 23 illustrates a handle 28 having at least one carrier attachment peg 29.

FIG. 24 is a top and front perspective view of a handle 28 having holding ridges 51.

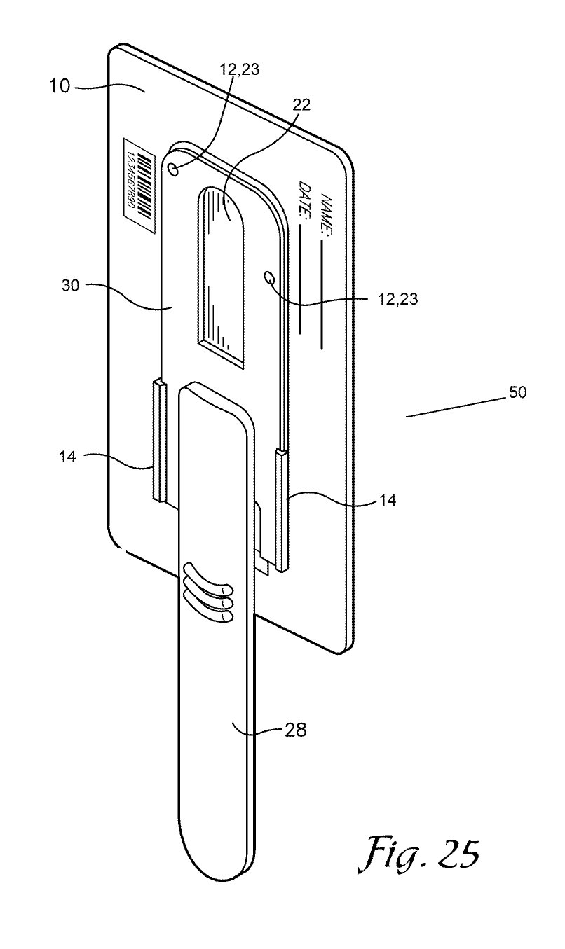

FIG. 25 is a top and front perspective view of a specimen collection device 50 (i.e., a specimen collector carrier 30 attached to a cassette 10). The specimen collector carrier 30 is attached to the cassette 10 by sliding the specimen collector carrier 30 into the at least one collector retaining flange 14, so that the at least one collector attachment peg 12 is aligned with the at least one cassette attachment void 23. The sample access void 27 of the specimen collector carrier 30 then aligns with the access void 15 within the receiver impression 13 of the cassette 10.

FIG. 34 is an alternative view of a specimen collection device 50 (i.e., specimen collector carrier 30 attached to a cassette 10) having barcode identification 18b on the handle 28 and the cassette 10, in addition to fingerprint identification 18c on the cassette 10, as an example of identification indicia.

FIG. 7 illustrates a protect apparatus 40 having a vertical orientation. The protect apparatus 40 comprises at least one protect flange 41 and protect attachment means 45. The protect apparatus 40 also has an identification void or window 44. In some aspects, the protect apparatus 40 includes ventilation means 43. In some aspects, the protect apparatus 40 includes at least one thumb indent 42.

FIG. 8 illustrates an alternative embodiment of a protect apparatus 40 having a horizontal orientation. The protect apparatus 40 comprises at least one protect flange 41 and protect at least one protect attachment means 45. In some aspects, the protect apparatus 40 includes at least one thumb indent 42.

FIG. 10 illustrates a protect apparatus 40 having a horizontal orientation and an identification void 44.

FIG. 11 illustrates a protect apparatus 40 having a vertical orientation and ventilation means 43.

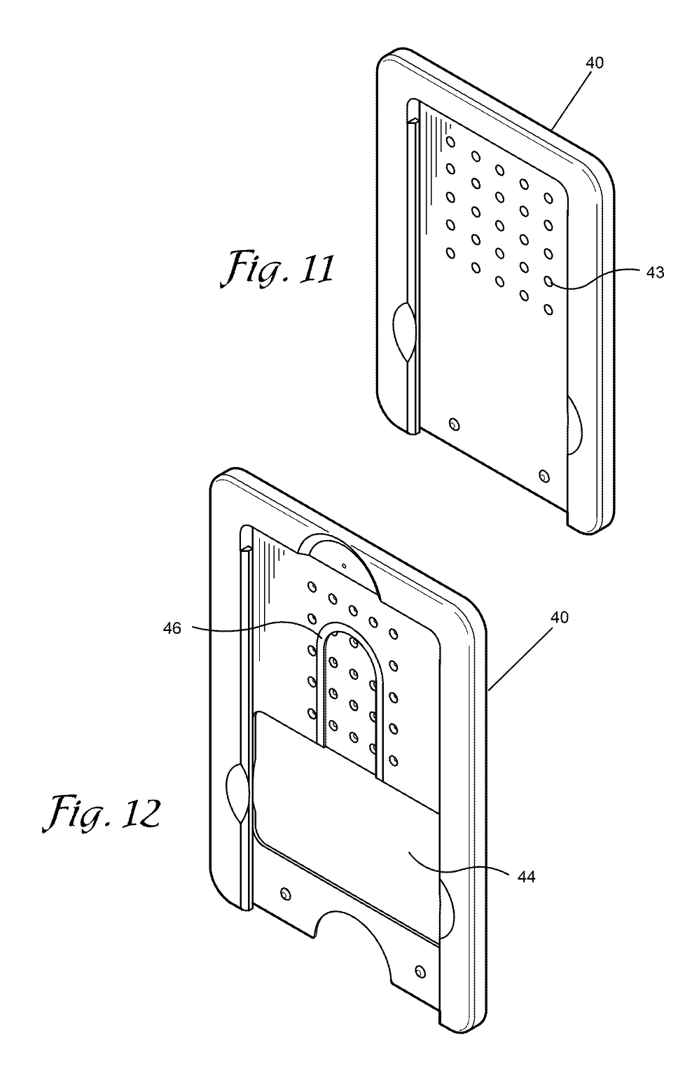

FIG. 12 illustrates a protect apparatus 40 having a vertical orientation and an identification void 44;

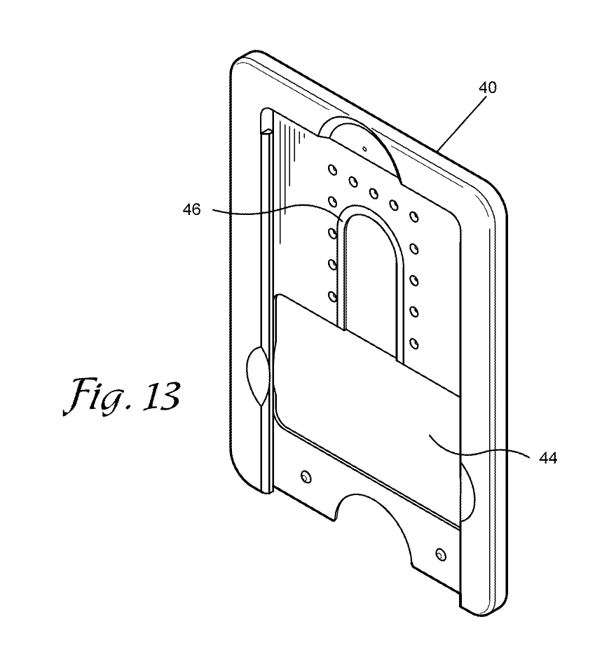

FIG. 13 illustrates a protect apparatus 40 having an identification void 44, and collection absorbent frame 46 to align with the collection absorbent 22 when the specimen collection device 50 is inserted into the protect apparatus.

FIG. 14 illustrates a specimen collection device 50, having a specimen collector carrier 30 (including a collection absorbent 22, cover 31 and handle 28), attached to a cassette 10 and the cassette 10 is attached to a protect apparatus 40.

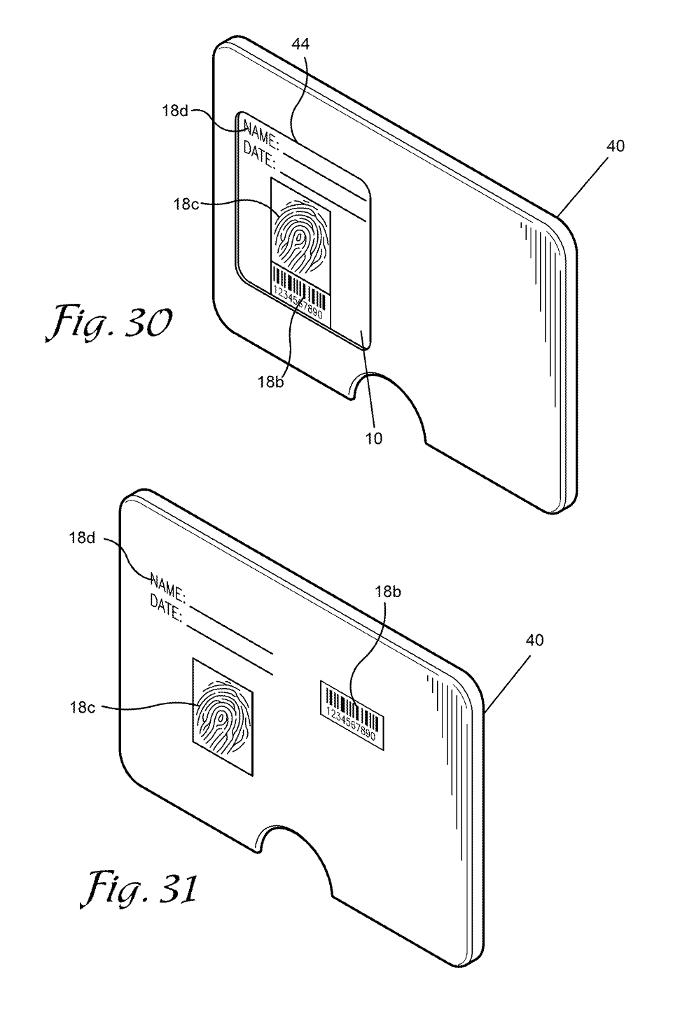

FIG. 30 illustrates a protect apparatus 40 having a specimen collection device 50 inserted such that the identification indicia (e.g., fingerprint identification 18c, barcode identification 18b, and handwritten identification 18d) located on the cassette 10 may be easily viewed without obstruction through the identification void 44 of the protect apparatus 40.

FIG. 31 illustrates a protect apparatus 40 having identification indicia on the rear portion of the protect apparatus, e.g., fingerprint identification 18c, barcode identification 18b, and handwritten identification 18d.

### DEFINITIONS

Unless defined otherwise, all technical and scientific terms used herein have the same meaning as is commonly understood by one of ordinary skill in the art. All patents, applications, published applications and other publications are incorporated by reference in their entirety. In the event that there is a plurality of definitions for a term herein, those in this section prevail unless stated otherwise.

As used herein, the term “about” designates that a value is within a statistically meaningful range. Such a range can be typically within 20%, more typically still within 10%, and even more typically within 5% of a given value or range. The allowable variation encompassed by the term “about” depends on the particular system under study and can be readily appreciated by one of ordinary skill in the art.

As used herein the term “biological sample” is used in its broadest sense and includes liquid or non-liquid samples or specimens from a wide variety of sources. Representative types of biological samples include tissue scrapings, whole blood, urine, cervical secretions, bronchial aspirates, sputum, saliva, feces, serum, synovial and cerebrospinal fluid, as well as laboratory preparations such as purified or partially purified macromolecules and cell culture materials. By way of example, the biological sample may be body fluid, body excretion, a population of cells, saliva, urine, mucus, tissue, or other biological sample type known in the art. Further examples of biological samples include, physiological/pathological body liquids (e.g., secretions, excretions, exudates and transudates) or cell suspensions (e.g., blood, lymph, synovial fluid, semen, saliva containing buccal cells, skin scrapings, hair root cells, etc.) of humans and animals; physiological/pathological liquids or cell suspensions of plants; liquid products, extracts or suspensions of bacteria, fungi, plasmids, viruses etc.; liquid products, extracts or suspensions of parasites including helminths, protozoas, spirochetes, etc.; liquid extracts or homogenates of human or animal body tissues (e.g., bone, liver, kidney, etc.); media from DNA or RNA synthesis; mixtures of chemically or biochemically synthesized DNA or RNA; and any other source in which DNA and/or RNA is or can be in a liquid medium. Preferably, the liquid containing the biological samples evaporates after applying the biological sample to the dry solid medium leaving macromolecules in dry form prior to subsequent analysis.

The term “entrain” means that during storage the macromolecules of a biological sample or evidence are bound to the collection absorbent without substantial reliance on ionic, covalent or van der Waals interactions.

The term “inert” is defined as molecules which have no deleterious interaction with macromolecules of interest within a sample and will not interfere with any subsequent analysis of the macromolecules.

Herein, the use of “or” means “and/or” unless stated otherwise. Furthermore, the use of the term “including”, as well as other forms such as “includes” and “included” is not limiting.

The term “sorb” means that the composition of the invention is absorbed, or otherwise incorporated into or onto the solid matrix in such a way as not to be readily removed from the matrix unless subjected to conditions which are intentionally or inadvertently performed to remove the sorbed composition from the solid matrix.

The terms “storing”, “storage”, “stored” and other derivatives of “store”, when referring to macromolecules including genetic material in dry form entrained to the collection absorbent, means the preservation of macromolecules in a form suitable for subsequent analysis and which has not undergone substantial degradation. The time period for which macromolecules, including genetic material, may be stored may be as short as the time necessary to transport a sample from the place of collection of the sample to the place where subsequent analysis is to be performed. The conditions under which the sample of macromolecules may be stored on the collection absorbent varies. Typically, samples are stored at temperatures from −200° C. to 40° C. In addition, stored samples may optionally be stored in dry or desiccated conditions or under an inert atmosphere. Storage may be for a few seconds up to many years, preferably, about 4 seconds up to 100 years or more.

As used herein, the term “subject” refers to a mammal, preferably a human, from which a biological sample is to be collected.

The term “weak base” includes a composition which has a pH of about 6 to 10, preferably about pH 8 to 9.5. One function of the weak base is to act as a buffer to maintain a composition pH of about 6 to 10, preferably about pH 8.0 to 9.5, for example, pH 8.6.

Unless defined otherwise, all technical and scientific terms used herein have the same meaning as is commonly understood by one of skill in the art to which this invention belongs at the time of filing. The meaning and scope of terms should be clear; however, in the event of any latent ambiguity, definitions provided herein take precedent over any dictionary or extrinsic definition. Further, unless otherwise required by context, singular terms shall include pluralities and plural terms shall include the singular.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Collection of Evidence at a Crime Scene

The user of the collection device 50 will retrieve the collection components from sterile primary packaging of the device. The user may attach the handle 28 to the specimen collector carrier 30. Any protective film or packaging covering the collection absorbent 22 may be removed using sterile techniques (e.g., wearing sterile gloves, handling with sterile precautions, etc.). The user may then contact the collection absorbent 22 with surfaces having potential evidence or biological samples for collection. If the user desires to collect a biological sample from an unwilling suspect, attaching the handle 28 to the specimen collector carrier 30 will aid in collecting a sample without endangering the appendages of the user. Once the biological sample or evidence has been collected, the user may attach the specimen collector carrier 30 to the cassette 10 by aligning the specimen collector carrier 30 between the collector retaining flanges 14 and sliding the specimen collector carrier 30 between the flanges such that the collection absorbent 22 aligns with the receiver impression 13 and rests over the access void 15. The cassette attachment voids 23 of the specimen collector carrier 30 should align with the collector attachment pegs 12 of the cassette 10. The user then may attach the specimen collector carrier 30 to the cassette 10 by pressing the pegs 12 into the attachment voids 23.

The user may then insert the specimen collection device 50 into a protect apparatus 40. The specimen collection device 50 is slid along the protect flanges 41 into place. The protect attachment means 45 located on the cassette 10 should be aligned with the protect attachment means 45 located on the protect apparatus 40 once the specimen collection device 50 is in place. The ventilation means 43 will allow the sample collected on the collection absorbent 22 to dry.

The user may fill in an identification form or identification card (FIGS. 35 and 36) that includes an identification indicia on the form identical to identification indicia located on the cassette 10. The identical identification indicia may also be located on the specimen collector carrier 30. The identification form and the collection device may then be submitted for further analysis at a laboratory.

Components of the collection device 50 and the information form may include areas where additional identification indicia may be added for identification means in the laboratory. Once the collection device is received by the analysis laboratory, the identification information associated with the cassette 10 may be entered into a database or computer system. The means of entering such information depends upon the identification indicia storage means. For instance, if the identification indicia is a bar code, the cassette may be scanned. If the identification indicia is a magnetic strip, the cassette may be swiped in a device that can read magnetic strip encoding.

The specimen collection device 50 may be removed from the protect apparatus 40. The user may grasp the protect apparatus 40 such that the user's appendages grasp the thumb indents 42 of the protect apparatus 40. Once removed from the protect apparatus 30, the specimen collection device 50 may be placed in a sample tray of an automated device or machine that punches samples from the collection absorbent 22. Typically, the punches will be collected in a vial. The vial or a plate containing the vial will be transferred to an automated sequencing machine and analyzed. The identification information is associated with the sample by the automated analysis devices such that the resultant readout information is associated with the correct identification information.

The invention illustratively disclosed herein suitably may be practiced in the absence of any element, which is not specifically disclosed herein. It is apparent to those skilled in the art, however, that many changes, variations, modifications, other uses, and applications to the method are possible, and also changes, variations, modifications, other uses, and applications which do not depart from the spirit and scope of the disclosure are deemed to be covered by the disclosure, which is limited only by the claims which follow.

What is claimed is:

1. A specimen collection device into which a collected biological sample obtained from a subject is secured by a user to maintain evidentiary chain of custody requirements while providing unobstructed access to the collected sample for automated analysis, the device comprising:
a. a specimen collector carrier comprising a collection absorbent securely connected to the specimen collector carrier; and
b. a cassette comprising a receiver impression aligned with at least one collector retaining flange, wherein the receiver impression is configured to align with the specimen collector carrier upon insertion of the specimen collector carrier into the at least one collector retaining flange.

2. The device of claim 1, wherein the cassette further comprises a tamper resistant attachment means for securing the specimen collector carrier to the cassette.

3. The device of claim 1, wherein the cassette further comprises an access void in unobstructed registration with the collection absorbent when the specimen collector carrier is attached to the cassette.

4. The device of claim 1, wherein the cassette further comprises a clean punch area.

5. The device of claim 1, wherein the specimen collector carrier further comprises a handle, wherein the handle comprises an attachment means for removably connecting the handle to the specimen collector carrier.

6. The device of claim 1, wherein the specimen collector carrier further comprises a cover removably attached to the specimen collector carrier through an attachment means.

7. The device of claim 1, wherein the device further comprises identification indicia.

8. The device of claim 1 further comprising a protect apparatus, wherein the protect apparatus comprises at least one protect flange and is configured to register with the shape of the cassette upon insertion of the cassette into the at least one protect flange.

9. The device of claim 8, wherein the protect apparatus further comprises identification indicia.

10. The device of claim 8, wherein the protect apparatus further comprises an identification window.

11. The device of claim 8, wherein the protect apparatus further comprises ventilation means.

12. The device of claim 8, wherein the protect apparatus further comprises thumb indents to facilitate the removal of the specimen collection device from the protect apparatus by the user.

13. A specimen collection device into which a collected biological sample obtained from a subject is secured to maintain evidentiary chain of custody requirements while providing unobstructed access to the collected sample for automated analysis, the device comprising:
a. a specimen collector carrier comprising a collection absorbent securely connected to the specimen collector carrier;
b. a handle removably connected to the specimen collector carrier, wherein the handle comprises an attachment means for removably connecting the handle to the specimen collector carrier, and wherein the handle permits manipulation of the specimen collector carrier during collection of the biological sample from the subject; and
c. a cassette comprising a receiver impression aligned with at least one collector retaining flange, wherein the receiver impression is configured to align with the specimen collector carrier upon insertion of the specimen collector carrier into the at least one collector retaining flange.

14. A specimen collection device into which a collected biological sample obtained from a subject is secured to maintain evidentiary chain of custody requirements while providing unobstructed access to the collected sample for automated analysis, the device comprising:
a. a specimen collector carrier comprising a collection absorbent securely connected to the specimen collector carrier;
b. a handle removably connected to the specimen collector carrier, wherein the handle comprises an attachment means for removably connecting the handle to the specimen collector carrier, and wherein the handle permits manipulation of the specimen collector carrier during collection of the biological sample from the subject;
c. a cassette comprising a receiver impression aligned with at least one collector retaining flange, wherein the receiver impression is configured to align with the specimen collector carrier upon insertion of the specimen collector carrier into the at least one collector retaining flange;
d. a cover for protecting the collection absorbent, wherein the cover comprises a means for removably attaching to the specimen collector carrier and permits a user to avoid directly contacting the collection absorbent; and
e. a protect apparatus comprising at least one protect flange and configured to register with the shape of the cassette upon insertion of the cassette into the at least one protect flange.

15. A method for collecting and analyzing a biological sample obtained from a subject for storage and subsequent automated analysis, the method comprising:
a. providing a specimen collector carrier comprising a collection absorbent;
b. contacting the collection absorbent with a biological sample from the subject;
c. connecting the specimen collector carrier containing the biological sample to a cassette comprising a receiver impression aligned with at least one collector retaining flange by sliding the specimen collector carrier into the at least one collector retaining flange and aligning the specimen collector carrier with the receiver impression to form a specimen collection device;
d. storing the specimen collection device for analysis of the biological sample.

16. The method of claim 15 further comprising the step of attaching a handle to the specimen collector carrier.

17. The method of claim 15 further comprising the step of inserting the specimen collection device into an automatic sampling device.

18. The method of claim 15 further comprising the step of attaching the cassette to a protect apparatus.
